(19) United States
(12) Patent Application Publication
(10) Pub. No.: US 2011/0184257 A1
(43) Pub. Date: Jul. 28, 2011

(54) DEVICE FOR DETERMINING AND/OR MONITORING THE MOISTURE CONTENT OF THE SKIN

(76) Inventors: Herbert Boll, Stuttgart (DE); Ulrich Ladstaetter, Weinstadt (DE)

(21) Appl. No.: 12/993,031

(22) PCT Filed: Apr. 27, 2009
(86) PCT No.: PCT/EP09/55067

§ 371 Date: Feb. 8, 2011

(30) Foreign Application Priority Data
Jun. 19, 2008 (DE) 10 2008 002 520.8

(51) Int. Cl. A61B 5/00

(52) U.S. Cl. 600/306

(57) ABSTRACT

A device for determining and/or monitoring the moisture content of the skin of a person or an animal, in particular for detecting dehydration in an elderly person or a person requiring care, as well as a corresponding method and use of such a device.

### FIELD OF THE INVENTION

The present invention relates to a device for determining and/or monitoring the moisture content of the skin of a person or an animal, in particular for detecting dehydration in an elderly person or a person requiring care as well as a corresponding method and the use of such a device.

### BACKGROUND INFORMATION

Fluid deficiency, also known as dehydration, is understood to be an increased loss of body fluid without a compensatory fluid intake. Fluid may be lost through the gastrointestinal tract, the exhaled air, the urine or the skin, for example, through perspiration or through major blood loss. A great deal of body fluid is lost in febrile diseases or with extreme physical exertion, but also a number of diseases such as kidney disorders may result in a severe fluid imbalance.

Dehydration of patients requiring care is a very serious problem in geriatrics, in particular in care of the elderly. Approximately 7% of the elderly are dehydrated on admission to the hospital, i.e., they have a fluid deficiency as their main or concomitant condition. Immobility, lack of thirst perception and not least of all poverty among the elderly are factors which may result in a fluid deficiency.

According to the care guidelines currently in effect, a patient's dehydration status is evaluated by assessing the mucous membranes, by a skin fold test in which a fold of skin is pinched up and the time until the fold relaxes is used as a measure of the dehydration, or by recording a log of fluid intake.

German Patent Application No. DE 10 2004 027 909 A1 describes a device for determining perspiration for testing antiperspirants, so-called deodorants, in which the device is attached by a patch, an adhesive strip or a piece of fabric. According to German Patent Application No. DE 10 2004 027 909 A1, the device has a moisture sensor, which is situated at a distance above the skin, to prevent falsification of the moisture measurement due to the moisture sensor coming in contact with liquid and salts. However, such a device does not make it possible to measure the moisture content of the skin because, as already explained, dehydration occurs when the factors of fluid loss and inadequate fluid intake occur at the same time, so no inferences about the existence and degree of dehydration are possible merely by measuring the fluid loss.

### SUMMARY

An example device according to the present invention and an example method according to the present invention may have the advantage that they enable both qualitative and quantitative determination of the moisture content of the skin, providing objective values for the patient and the nursing staff, so that dehydration of persons requiring care in geriatrics is detected reliably and at an early point in time, attention is drawn to dehydration or the risk of dehydration, and therefore the burden on the nursing staff is significantly reduced. Furthermore, the production costs for the example device according to the present invention may be kept extremely low, so that the device according to the present invention may be used as a disposable product. Furthermore, the device according to the present invention has a high wear comfort and may remain attached to the skin for several days.

In accordance with the present invention, an example device is provided for determining and/or monitoring the moisture content, in particular the moisture content of the skin of a person or animal. The example device according to the present invention as well as the example method according to the present invention, which is explained later, are used in particular for detecting dehydration in a person or animal. Within the scope of the present invention, this involves in particular an elderly person or a person requiring care. The example device according to the present invention and also the example method according to the present invention, which is explained later, are advantageously suitable for ensuring a qualitative and quantitative determination of the moisture content of the skin.

The example device according to the present invention includes a sensor for measuring the moisture content of the skin and fastener for detachable attachment of the device to the body of a person or animal and a signal generator for outputting one or more measuring results, in particular evaluated results, and/or one or more warning signals, and/or a memory means for saving one or more measuring results, in particular evaluated results. According to the present invention, the sensor is situated in such a way that it contacts the skin when the device is attached.

The sensor is able to detect the moisture content of skin electrically, in particular by a resistance measurement, optically, in particular spectroscopically, and/or chemically, in particular by measuring the pH value.

The example device according to the present invention may have a voltage and/or current supply device for supplying voltage and/or current to the sensor, the signal generator, the memory element, and additional electronic devices, for example, a logic means and/or a temperature measuring device.

Within the scope of a specific embodiment of the present invention, the sensor has two electrodes which contact the skin when the device is attached. The electrodes are connected either to a voltage and/or current supply device (5) and to a device for voltage, current, and/or resistance measurement or to a combination device for voltage and/or current supply and voltage, current, and/or resistance measurement.

The sensor may be operated with either direct current or alternating current. To measure the moisture content of the skin on the basis of a resistance measurement, it may be advantageous to supply alternating current to the electrodes in order to avoid an electrolysis-induced falsification of the measuring results in a long-term measurement. The voltage and/or current supply device and/or the combination device for voltage and/or current supply and voltage, current, and/or resistance measurement therefore preferably supplies an alternating current to the electrodes.

Within the scope of the specific embodiment according to the present invention in particular, it may be advantageous to supply the electrodes with an alternating current of a frequency of ≧1 Hz to ≦10 MHz because an alternating current of this frequency range often makes it possible to penetrate into deeper layers of the body, in particular layers of the skin.

Within the scope of a further specific embodiment of the present invention, the sensor has a radiation source and a radiation sensor.

Within the scope of a first design of this specific embodiment, the radiation source and the radiation sensor are designed and situated in such a way that the radiation emitted by the radiation source penetrates through the skin and the radiation remaining after passing through the skin is measured by the radiation sensor. Within the scope of the present invention, the term “radiation” is understood to be electromagnetic radiation.

Within the scope of a second design of this specific embodiment, the radiation source and the radiation sensor are designed and situated in such a way that the radiation emitted by the radiation source is reflected on a layer of skin, in particular an internal layer, and the reflected radiation is measured by the radiation sensor.

The moisture content, in particular the moisture content of skin, may be determined advantageously from the measuring result of the radiation sensor, in particular with a knowledge of the emission spectrum of the radiation source, because liquids, in particular water, absorb radiation of a [given] wavelength differently as a function of the quantity of fluid through which it passes, in particular the quantity of water.

The wavelength or the dominant wavelength of the radiation source preferably corresponds to a wavelength at which absorption by water is high. For example, water, among other substances, has a high absorption in a wavelength range of ≧2700 to ≦3700 nm.

In selecting the wavelength or the dominant wavelength of the radiation source, another factor to preferably take into account is that body components, in particular components of the skin other than water, have the lowest possible absorption at this wavelength and/or in this wavelength range.

To prevent damage to tissue, long-term radiation at a wavelength of <280 nm, for example, ≦320 nm, in particular ≦400 nm should be avoided. In the case of short, e.g., pulsed, radiation, however, it is nevertheless possible within the scope of the present invention to use radiation from the ultraviolet spectral range.

Since the absorption of water is low in a wavelength range of >400 nm to <950 nm, the wavelength or dominant wavelength of the radiation source is ≧950 nm, in particular ≧1000 nm, within the scope of one specific embodiment of the present invention.

Within the scope of the present invention, the fastener may generally include a piece of fabric, in particular a textile, and/or a strip of adhesive tape, in particular a patch. Within the scope of a preferred specific embodiment of the present invention, the fastener is a patch, in particular an adhesive patch.

Within the scope of the present invention, the signal generator may be or include a discolorable material, for example, a material that is discolorable due to a change in pH, voltage and/or temperature, in particular an indicator paper and/or a luminous means and/or a display, in particular an LCD display and/or a transmitter, for example, an infrared transmitter, a Bluetooth transmitter or a radio frequency transmitter.

The memory element may be readable by an external reader device. The memory element may be, for example, an RFID (radio frequency identification) chip. Advantageously the data thereby measured and saved may be read out by an appropriate reader device.

Within the scope of another preferred specific embodiment of the present invention, the device according to the present invention also has logic element, for example, a microprocessor for evaluating the measured results obtained by the moisture content sensor and/or other sensors, for example, a temperature sensor. The element preferably relay the evaluated measuring results to the signal generator and/or to the memory element and/or activate the signal generator.

The example device according to the present invention is preferably designed to be monolithic. The term “monolithic” means that the elements of the device according to the present invention form a unit, in particular a cohesive unit. A monolithic design of the device advantageously reduces the risk of destruction of the device and injury to the patient or the nursing staff.

The electronics, in particular the electronic components, of the device according to the present invention may be designed as film electronics, in particular flexible film electronics within the scope of a preferred specific embodiment of the present invention. Electric lines and/or the signal generator and/or the memory element and/or the voltage and/or current supply device and/or the combination device for voltage and/or current supply and voltage, current and/or resistance measurement and/or the radiation source and/or the radiation sensor and/or the logic element may preferably be situated in and/or on a film of the fastener, in particular an adhesive film. In particular, the electric lines and/or the transmitter and/or the memory element and/or the voltage and/or current supply device and/or the combination device for voltage and/or current supply and voltage, current and/or resistance measurement and/or the radiation source and/or the radiation sensor and/or the logic element may be situated on a film of the fastener, in particular a plaster film. The example embodiment of the electronics as film electronics has the advantage that, on the one hand, it reduces production costs and thus allows its use as a disposable product. On the other hand, wear comfort may be greatly increased by using flexible film electronics.

Within the scope of another preferred specific embodiment of the present invention, the device according to the present invention also has a temperature sensor. Using a temperature sensor advantageously offers the possibility of performing a plausibility check on the measured moisture values and/or correcting these values by using temperature-related factors.

Another object of the present invention is to provide a method for determining and/or monitoring the moisture content, in particular the moisture content of the skin of a person or animal using a device according to the present invention, in which the moisture content of the skin is measured by the moisture content sensor and is output by the signal generator and/or stored, in particular buffered, in the memory element.

The moisture content sensor preferably measures the moisture content of the skin at intervals, in particular at regular intervals of more than one hour, for example. As already explained in conjunction with the example device according to the present invention, the measuring results of the moisture content sensor may be evaluated by the logic element and then stored, in particular buffered, in the memory element. Within the scope of a preferred specific embodiment, the signal generator outputs the moisture content when there is a critical change, in particular a reduction, and/or outputs a warning signal when a critical threshold is exceeded. This may occur, for example, by the logic element switching a certain voltage output and/or current output, which activates the signal generator and/or the memory element, in particular the signal generator, when there is a critical change, in particular a reduction in the moisture content, and/or when a critical threshold value is exceeded. For example, by switching the output, an indicator paper may be discolored or a display or a luminous means or a radio frequency transmitter cooperating with a receiving system may be activated, or the measured value may be stored in a RFID chip.

Another object of the present invention is the use of a device according to the present invention in medicine, in particular in geriatric medicine, for detection of dehydration of a person, in particular someone elderly and/or requiring care.

### BRIEF DESCRIPTION OF THE DRAWINGS

Additional advantages and advantageous embodiments of the object according to the present invention are illustrated by the FIGURE. It should be pointed out that the FIGURE has only a descriptive character and is not intended to restrict the present invention in any form.

FIG. 1 shows a highly schematic representation of an example device according to the present invention.

### DETAILED DESCRIPTION OF EXAMPLE EMBODIMENTS

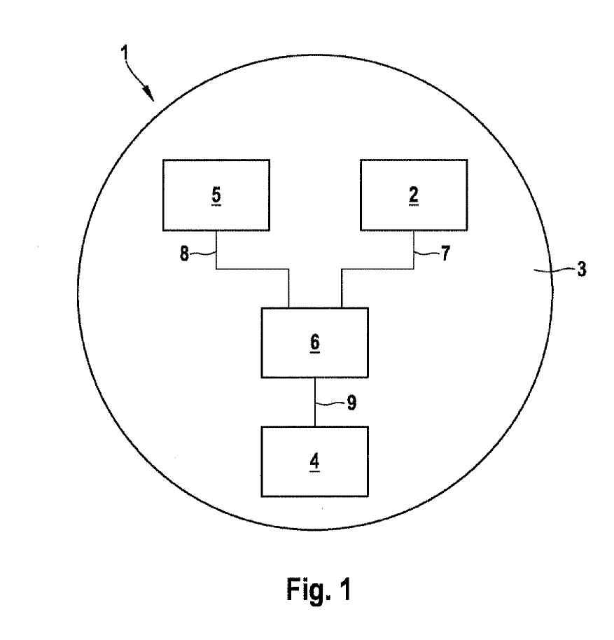

FIG. 1 shows that this specific embodiment of a device 1 according to the present invention for determining and/or monitoring the moisture content of the skin of a person includes a sensor 2 for measuring the moisture content of the skin and fastener 3 for detachable attachment of device 1 to the body of a person, and a signal generator 4 for outputting one or more measuring results and/or one or more warning signals, a voltage and/or current supply device 5, and logic means 6. Within the scope of the specific embodiment illustrated in FIG. 1, fastener 3 is designed as a patch. The patch has several adhesive areas (not shown) facing the body for detachably attaching device 1 to the body. Moisture content sensor 2 has a signal conducting connection to logic means 6 via a signal line 7, the logic element including a microprocessor. Logic element 6 receives electricity from voltage and/or current supply device 5 via a power supply line 8, so that the electricity is relayed by logic element 6 over line 7 and another line 9 to supply power to moisture content sensor 2 of signal generator 4.

1-12. (canceled)

13. A device for determining or monitoring moisture content of skin of a person or animal for detecting dehydration, comprising:
a sensor to measure the moisture content of the skin;
a patch for detachable attachment of the device to a body of the person or animal; and
a signal generator to output at least one of: i) at least one measuring result, and ii) at least one warning signal;
wherein the sensor contacts the skin when attached thereto.

14. A device for determining or monitoring moisture content of skin of A person or animal for detecting dehydration, comprising:
a sensor to measure the moisture content of the skin;
a patch for detachable attachment of the device to a body of the person or animal; and
a memory element to store at least one measuring result;
wherein the sensor contacts the skin when attached thereto.

15. The device as recited in claim 13, wherein the sensor detects the moisture content of the skin at least one of: i) electrically by a resistance measurement, ii) optically via spectroscopy, and iii) chemically by measuring a pH value.

16. The device as recited in claim 13, wherein the sensor has two electrodes which contact the skin when attached thereto and are connected to i) at least one of a voltage supply device and a current supply device, and ii) a device for one of voltage, current, and resistance measurement.

17. The device as recited in claim 13, wherein the sensor has two electrodes which contact skin when attached thereto and are connected to a combination device for one of voltage supply, current supply and resistance measurement.

18. The device as recited in claim 16, wherein at least one of the voltage supply device and current supply device supplies an alternating current to the electrodes.

19. The device as recited in claim 17, wherein the combination device supplies an alternating current to the electrodes.

20. The device as recited in claim 13, wherein the sensor includes a radiation source and a radiation sensor, which are designed and situated in such a way that radiation emitted by the radiation source passes through the skin, and the radiation remaining after passing through the skin is measured by the radiation sensor

21. The device as recited in claim 14, wherein the sensor includes a radiation source and a radiation sensor, which are designed and situated in such a way that radiation emitted by the radiation source is reflected on a layer of the skin, and the reflected radiation is measured by the radiation sensor.

22. The device as recited in claim 13, wherein the signal generator includes at least one of: i) a material discolorable due to a change in at least one of pH value, voltage, and temperature, ii) an indicator paper, iii) a display, iv) an LCD, v) an infrared transmitter, vi) a Bluetooth transmitter, and vii) a radio frequency transmitter.

23. The device as recited in claim 14, wherein the memory element is adapted to be read out by an external reader device.

24. The device as recited in claim 23, wherein the memory element is an RFID chip.

25. The device as recited in claim 13, further comprising:
a processor to evaluate the measuring results detected by the sensor.

26. The device as recited in claim 13, wherein electronic components of the device are embodied as film electronics.

27. The device as recited in claim 13, wherein the film electronics are flexible film electronics.

28. A method for determining and monitoring moisture content of skin of a person or animal, comprising:
attaching a device to the person or animal, the device including a sensor to measure the moisture content of the skin, a patch for detachable attachment of the device to a body of the person or animal and a signal generator to output at least one of: i) at least one measuring result, and ii) at least one warning signal, wherein the sensor contacts the skin when attached thereto;
measuring by the sensor the moisture content of the skin; and
outputting by the signal generator, the moisture content.

29. A method for determining and monitoring moisture content of skin of a person or animal, comprising:
attaching a device to the person or animal, the device including a sensor to measure the moisture content of the skin, a patch for detachable attachment of the device to a body of a person or animal and a signal generator to output at least one of: i) at least one measuring result, and ii) at least one warning signal, wherein the sensor contacts the skin when attached thereto;
measuring by the sensor the moisture content of the skin; and
saving the moisture content in the memory device.

30. The method as recited in claim 28, wherein the signal generator outputs a warning signal at least one of: when there is a critical change in the moisture content, and when a critical threshold value is exceeded.

31. A method for determining and monitoring moisture content of skin of a person for detecting dehydration of the person, comprising:
attaching a device to the person or animal, the device including a sensor to measure the moisture content of the skin, a patch for detachable attachment of the device to a body of the person and a signal generator to output at least one of: i) at least one measuring result, and ii) at least one warning signal, wherein the sensor contacts the skin when attached thereto;
measuring by the sensor the moisture content of the skin; and
outputting by the signal generator, the moisture content for detecting dehydration of the person.
